(19) United States
(12) Patent Application Publication
(10) Pub. No.: US 2017/0233828 A1
(43) Pub. Date: Aug. 17, 2017

(54) GLYCOSYLTRANSFERASE GENE EXPRESSION PROFILE TO IDENTIFY MULTIPLE CANCER TYPES AND SUBTYPES

(71) Applicant: University of Cape Town, Rondebosch, Cape Town (ZA)

(72) Inventors: Kevin Jonathan NAIDOO, Claremont (ZA); Jahanshah ASHKANI, Plattekloof (ZA)

(73) Assignee: University of Cape Town, Rondebosch, Cape Town (ZA)

(21) Appl. No.: 15/519,344

(22) PCT Filed: Oct. 15, 2015
(86) PCT No.: PCT/IB2015/057916

§ 371 Date: Apr. 14, 2017

(30) Foreign Application Priority Data
Oct. 15, 2014 (GB) 1418242.2

(51) Int. Cl. C12Q 1/68; G06F 19/20

(52) U.S. Cl. CPC C12Q 1/6886; G06F 19/20; C12Q 2600/158; C12Q 2600/112

(57) ABSTRACT

This invention relates to a method of classification of cancer type or subtype in a subject by detecting glycosyltransferase gene expression in a sample from the subject. In particular, where the method is for classification of cancer subtype in the subject the glycosyltransferase genes may be survival associated glycosyltransferase genes. The invention further relates to a kit comprising glycosyltransferase gene biomarkers for use with the method, and a method of treatment of cancer in a subject with the use of the method or kit of the invention.

### FIELD OF THE INVENTION

This invention relates to a method of identification of cancer types in a subject comprising the use of supervised algorithms that have been trained to evaluate quantified glycosyltransferase gene expression in a sample from the subject. In addition, the method relates to a method of cancer subtype classification and/or identification of a cancer subtype within a specific cancer type comprising the use of unsupervised and supervised algorithms that have been trained to evaluate quantified glycosyltransferase gene expression. The invention further relates to a kit comprising glycosyltransferase gene capture probes or primers for use with the method, and a method of treatment of cancer comprising the use of the method and/or kit of the invention to select an appropriate treatment regime for a subject and/or to monitor the response of the subject to the cancer treatment.

### BACKGROUND OF THE INVENTION

Cancer is one of the most dreadful diseases of this century, which can be life threatening. Early diagnosis and treatment relevant to the cancer type may improve the prognosis for cancer patients. The molecular characteristic of each type or subtype of cancer affects its sensitivity to treatments, which signifies the importance of precise prognosis in maximizing the efficacy and minimizing the toxicity of treatments. Despite the impact of the use of global gene expression analysis by genomic and epigenomic studies using high-throughput technologies, in elucidating the molecular characteristics of cancer, a broad diversity is often observed in biological behaviour, clinical prognosis, sensitivity to treatments, and therapeutic targets. This highlights the importance of characterising these possible subclasses within a cancer type. Such classification in the context of gene expression may be achievable through identification of key drivers in cancer biology.

Tumour cells have an altered cellular biochemistry that facilitates evasion of growth suppressors and apoptosis as well as acquire invasive, angiogenic and metastatic properties. More pointedly it is unclear to what extent the degree of specific glycosylation mechanisms interface with these pathways. It is known that changes occur in glycans and including glycans on proteins and lipids in and on cancer cells, making these altered glycans promising candidates for use as cancer biomarkers that may be used for early diagnosis, monitoring and therapy in a cancer patient. However, due to the lack of suitable analytical methods for glycan-biomarker identification and detection most of the existing cancer markers are the glycosylated proteins themselves that are not recommended for early detection because of poor sensitivity and specificity.

Furthermore, despite progress in feature selection algorithms used in different cancer subtyping methods, the identification of driver genes involved in different types of cancers remains challenging. Therefore, the development of novel strategies are significantly needed for improved early diagnosis and efficient cancer therapy.

### SUMMARY OF THE INVENTION

According to a first embodiment of the invention, there is provided a method of identification of cancer type in a subject by obtaining a glycosyltransferase gene expression profile from a set of glycosyltransferase genes in a test sample from the subject and comparing the gene expression profile from the test sample to a reference set of glycosyltransferase gene expression centroids from known cancer types, wherein the subject's cancer type is identified as the cancer type corresponding to the nearest reference set glycosyltransferase gene centroid compared with the glycosyltransferase gene expression profile from the test sample.

The method may comprise the following steps:
(a) obtaining a glycosyltransferase gene expression profile from a set of glycosyltransferase genes in a test sample from the subject, wherein the glycosyltransferase genes are selected from the group consisting of the glycosyltransferase genes listed in Table 1;
(b) calculating the distance of the gene expression profile of the test sample to each of a set of reference glycosyltransferase gene expression centroids from known cancer types; and
(c) assigning the test sample to a cancer type wherein the cancer type is determined as that corresponding to the nearest reference set glycosyltransferase gene centroid compared with the glycosyltransferase gene expression profile from the test sample.

The method may further comprise the following steps:
(a) obtaining a glycosyltransferase gene expression profile for each of a training set of cancer samples from subjects with known cancer types, wherein the glycosyltransferase genes are selected from the group consisting of the glycosyltransferase genes listed in Table 1;
(b) using a supervised algorithm to construct gene expression centroids for each of the cancer types in the training set;
(c) obtaining a glycosyltransferase gene expression profile from a set of glycosyltransferase genes in a test sample from the subject, wherein the glycosyltransferase genes are selected from the group consisting of the glycosyltransferase genes listed in Table 1;
(d) calculating the distance of the gene expression profile of the test sample to each of glycosyltransferase gene expression centroids from the training set of known cancer types; and
(e) assigning the test sample to a cancer type wherein the cancer type is determined as that corresponding to the nearest training set glycosyltransferase gene centroid compared with the glycosyltransferase gene expression profile from the test sample.

The glycosyltransferase genes may consist of any 5 or more, any 10 or more, any 20 or more, any 30 or more, any 40 or more, any 50 or more, any 60 or more, any 63 or more, any 70 or more, any 80 or more, any 90 or more, any 100 or more, any 110 or more any 120 or more any 130 or more, any 140 or more any 150 or more, any 160 or more any 170 or more, any 177 or more, optionally any 178 or more, further optionally any 179 or more, still further optionally any 180 or more, 181 or more, 182 or more, 183 or more, 184 or more, 185 or more etc. and up to still further optionally all 210 of the glycosyltransferase genes listed in Table 1.

Preferably, the glycosyltransferase genes consist of all 210 of the glycosyltransferase genes listed in Table 1.

In particular, where all of the 210 glycosyltransferase genes are used, the method of the invention may be used to identify cancer types selected from breast, including breast invasive carcinoma, brain, including glioblastoma multiforme, colon, including colon adenocarcinoma, kidney, including kidney renal clear cell carcinoma, lung, including lung squamous cell carcinoma, and ovarian, including ovarian serous cystadenocarcinoma cancer.

Where at least 50 glycosyltransferase genes are used, the method of the invention may be used to identify cancer types selected from breast, including breast invasive carcinoma, brain, including glioblastoma multiforme, colon, including colon adenocarcinoma, and ovarian, including ovarian serous cystadenocarcinoma cancer.

Where at least 5 glycosyltransferase genes are used, the method of the invention may be used to identify cancer types selected from brain, including glioblastoma multiforme, and ovarian, including ovarian serous cystadenocarcinoma cancer.

The reference glycosyltransferase gene expression centroids or training set of glycosyltransferase genes from known cancer types, or both may comprise or optionally consist of cancer types selected from breast, including breast invasive carcinoma, brain, including glioblastoma multiforme, colon, including colon adenocarcinoma, kidney, including kidney renal clear cell carcinoma, lung, including lung squamous cell carcinoma, and ovarian, including ovarian serous cystadenocarcinoma cancer.

The sample may be any bodily sample, such as, but not limited to, a tissue, blood, plasma or serum sample.

Step (a) of the method is preferably performed with cDNA or mRNA amplified from RNA extracted from the sample. Many means of amplification of cDNA or mRNA from RNA templates are known to those skilled in the art and many commercial kits for RNA extraction and amplification of cDNA and mRNA are available.

The determining or quantification of expression levels in step (a) may be performed using a dot blot procedure well known to those skilled in the art, including but not limited to, membrane-based Northern blot or Southern blot, or by means of a miniaturised dot blot procedure such as a microarray. Many commercial kits for use in such procedures are available to those skilled in the art.

Alternatively, the quantification may be performed by means of a quantitative polymerase chain reaction (PCR) method. Different methods for quantitative PCR of both RNA and DNA are known to those skilled in the art and commercial kits for use in such procedures are available. Examples of such procedures include, but are not limited to, quantitative reverse transcriptase PCR (RT-qPCR) which may or may not be real time.

In one particular embodiment of the invention, the method may comprise the steps of:
(a) purifying total RNA from the sample of the subject;
(b) converting the RNA into cDNA by reverse transcription and labelling the cDNA with a fluorescent dye or amplifying mRNA from the RNA and labelling the mRNA with a fluorescent dye;
(c) contacting a microarray slide printed with target oligonucleotide probes specific for the glycosyltransferase genes or a gene fragment of each selected from the group consisting of the glycosyltransferase genes listed in Table 1 with the fluorescently labelled cDNA or mRNA;
(d) hybridizing the fluorescently labelled cDNA or mRNA to the target oligonucleotide probes;
(e) quantifying the intensity of fluorescence of cDNA or mRNA bound to their target oligonucleotide probes on the microarray slide, whereby the intensity of fluorescence at each target corresponds to the relative abundance of mRNA transcript for each subject glycosyltransferase gene, that is, the subject sample glycosyltransferase gene expression profile;
(f) calculating the distance of the gene expression profile of the subject sample to each of a set of glycosyltransferase gene expression centroids from known cancer types, wherein the glycosyltransferase gene expression centroid genes are selected from the group consisting of the glycosyltransferase genes listed in Table 1; and
(g) assigning the subject sample to a cancer type wherein the cancer type is determined as that corresponding to the nearest glycosyltransferase gene expression centroid compared with the glycosyltransferase gene expression profile from the subject sample.

In an alternative particular embodiment of the invention, the method may comprise the steps of:
(a) purifying total RNA from the sample of the subject;
(b) converting the RNA into cDNA by reverse transcription and labelling the cDNA with a fluorescent dye or a chromogenic substrate, or amplifying mRNA from the RNA and labelling the mRNA with a fluorescent dye or a chromogenic substrate;
(c) contacting a dot blot printed with target oligonucleotide probes specific for the glycosyltransferase genes or a gene fragment of each selected from the group consisting of the glycosyltransferase genes listed in Table 1 with the labelled cDNA or mRNA;
(d) hybridizing the labelled cDNA or mRNA to the target oligonucleotide probes;
(e) quantifying the intensity of the hybridized labelled cDNA or mRNA bound to their target oligonucleotide probes on the dot blot, whereby the intensity of the label at each target corresponds to the relative abundance of mRNA transcript for each gene, that is, the subject sample glycosyltransferase gene expression profile;
(f) calculating the distance of the gene expression profile of the subject sample to each of a set of glycosyltransferase gene expression centroids from known cancer types; and
(g) assigning the subject sample to a cancer type wherein the cancer type is determined as that corresponding to the nearest glycosyltransferase gene expression centroid compared with the glycosyltransferase gene expression profile from the subject sample.

In a further alternative particular embodiment of the invention, the method may comprise the steps of:
(a) purifying total RNA from the sample of the subject;
(b) amplifying cDNA from the RNA by RT-qPCR with specific primers for the glycosyltransferase genes or a gene fragment of each selected from the group consisting of the glycosyltransferase genes listed in Table 1;
(c) quantifying the amplified cDNA;
(d) assessing the relative abundance of the amplified cDNA of each of the genes, whereby the abundance of the amplified cDNA corresponds to the relative abundance of mRNA transcript for each gene, that is, the subject sample glycosyltransferase gene expression profile;
(e) calculating the distance of the gene expression profile of the subject sample to each of a set of glycosyltransferase gene expression centroids from known cancer types; and
(f) assigning the subject sample to a cancer type wherein the cancer type is determined as that corresponding to the nearest glycosyltransferase gene expression centroid compared with the glycosyltransferase gene expression profile from the subject sample.

The method may further comprise a step of classifying the subtype of the cancer in a subject with known cancer type, wherein the subtype classification comprises the steps of:
(a) obtaining a test glycosyltransferase gene expression profile from a cancer sample from a subject with known cancer type, wherein the glycosyltransferase genes are selected from the group consisting of the glycosyltransferase genes listed in Table 1;
(b) providing a reference set of glycosyltransferase gene expression profiles from subtypes of the corresponding cancer to that of the subject, wherein the glycosyltransferase genes are selected from the group consisting of the glycosyltransferase genes listed in Table 1;
(c) performing unsupervised clustering analysis on the test glycosyltransferase gene expression profile and the reference set of glycosyltransferase gene expression profiles, thereby grouping the test glycosyltransferase gene expression profile and the reference glycosyltransferase gene expression profiles into one or more subtypes.

Preferably, the cancer type of the subject may previously have been identified by means of the method of the invention.

Optionally, the method of subtyping may further comprise the following steps:
(d) obtaining a glycosyltransferase gene expression profile from a set of glycosyltransferase genes in a test sample from a subject with known cancer type, wherein the glycosyltransferase genes are selected from the group consisting of the glycosyltransferase genes listed in Table 1;
(e) calculating the distance of the gene expression profile of the test sample to each of a set of reference glycosyltransferase gene expression centroids from subtypes of the corresponding cancer to that of the subject; and
(f) assigning the test sample to a cancer subtype wherein the cancer subtype is determined as that corresponding to the nearest reference set glycosyltransferase gene centroid compared with the glycosyltransferase gene expression profile from the test sample.

Further optionally, the method may further comprise the following steps:
(f) obtaining a glycosyltransferase gene expression profile for each of a training set of cancer samples from subjects with a known cancer type, wherein the glycosyltransferase genes are selected from the group consisting of the glycosyltransferase genes listed in Table 1;
(g) using a supervised algorithm to construct gene expression centroids for each of the cancer types in the training set;
(h) obtaining a glycosyltransferase gene expression profile from a set of glycosyltransferase genes in a test sample from the subject, wherein the glycosyltransferase genes are selected from the group consisting of the glycosyltransferase genes listed in Table 1;
(i) calculating the distance of the gene expression profile of the test sample to each of glycosyltransferase gene expression centroids constructed in step (b); and
(j) assigning the test sample to a cancer subtype wherein the cancer subtype is determined as that corresponding to the nearest training set glycosyltransferase gene centroid compared with the glycosyltransferase gene expression profile from the test sample.

Survival information may be known for the reference or training set of glycosyltransferase gene expression profiles, in which case the method may optionally include a step of identifying the survival prognosis of the subject based on the survival prognosis of the subtype cluster within which the test glycosyltransferase gene expression profile groups.

In particular, the step of identifying the subtype of the cancer after identification of cancer type may be performed on samples from subjects identified as having breast, brain or ovarian cancer.

According to a further aspect of the invention there is provided a kit for identifying cancer type and optionally subtype in a subject according to the method of the invention, the kit comprising:
(a) primers for amplification of cDNA or mRNA;
(b) glycosyltransferase gene specific oligonucleotide probes for hybridizing to cDNA or mRNA in a dot blot procedure; or
(c) glycosyltransferase gene specific oligonucleotide probes printed on a microarray slide, to which labelled cDNA or RNA can be hybridized in a microarray method;
(d) an indicator means; and
(e) optionally, directions for use.

The dot blot procedure used may be a cDNA or mRNA dot blot procedure. Preferably, the procedure is a miniaturised dot blot such as a microarray.

In an alternative embodiment of the invention, the kit comprises:
(a) glycosyltransferase gene specific primers for amplification of cDNA or mRNA by quantitative real-time PCR; and
(b) an indicator means; and
(c) optionally, directions for use.

The kit may further comprise a reference set of glycosyltransferase polynucleotides from known cancer types or subtypes in a specific cancer type.

The primers may be sequence-specific primers, oligo(dT) primers or random primers. For example, the sequence-specific primers may be glycosyltransferase gene or gene fragment specific primers. Various methods and websites for primer design and labelling are well known to those skilled in the art. Furthermore, methods and kits for amplification of cDNA or mRNA are also well known and available to those skilled in the art.

The oligonucleotide probes may be DNA, RNA or a modified or derivative oligonucleotide thereof, typically with a minimum of 25 complementary bases to the target glycosyltransferase gene sequence. Methods for design and production of such oligonucleotide probes and for performance of Northern or Southern blotting and microarray procedures are well known to those skilled in the art.

Various methods for modification of oligonucleotides by 3′, 5′ or sequence modification are known to those skilled in the art including, but not limited to amino-modification, carboxy-modification, thiol-modification, aldehyde-modification, chemical phosphorylation modification, spacer, linker or dendrimer modification and many other methods.

The indicator means may be any indicator means known to those skilled in the art including but not limited to a chromogenic, radiolabel or fluorescent indicator molecule. Typically, the indicator means is incorporated into the cDNA or mRNA of step (a) during the amplification step.

Many well-known methods and kits for labelling of cDNA or mRNA during amplification are known to those skilled in the art.

The kit may also comprise computer readable instructions for any one or more of:
(a) determining the subject sample glycosyltransferase gene expression profile;
(b) identifying cancer type by:
   i. calculating the distance of the gene expression profile of the subject sample to each of a set of glycosyltransferase gene expression centroids from known cancer types; and
   ii. assigning the subject sample to a cancer type wherein the cancer type is determined as that corresponding to the nearest glycosyltransferase gene expression centroid compared with the glycosyltransferase gene expression profile from the subject sample; and
(c) classifying and/or identifying cancer subtype by:
   i. performing unsupervised consensus clustering analysis of the glycosyltransferase gene expression profile from the subject having known cancer type compared with glycosyltransferase gene expression profiles from a set of reference or training glycosyltransferase genes from subtypes of the corresponding cancer to that of the subject;
   ii. calculating the distance of the gene expression profile of the subject sample to each of a set of glycosyltransferase gene expression centroids from subtypes of the corresponding cancer to that of the subject; and
   iii. assigning the subject sample to a cancer subtype wherein the cancer subtype is determined as that corresponding to the nearest glycosyltransferase gene expression centroid compared with the glycosyltransferase gene expression profile from the subject sample.

Survival information may be known for the reference or training set of glycosyltransferase gene expression profiles or for the glycosyltransferase gene expression centroids from subtypes of the corresponding cancer to that of the subject, in which case the method may optionally include computer readable instructions for identifying the survival prognosis of the subject based on the survival prognosis of the subtype cluster within which the test glycosyltransferase gene expression profile groups.

According to a further aspect of the invention there is provided the use of the method of the invention in the selection of a treatment regime for a subject suspected of having, or known to have cancer comprising performing the method of the invention on a sample from the subject and then selection of the appropriate treatment regime for the cancer type or subtype identified in the subject.

The method of the invention may be performed prior to a subject undergoing therapeutic treatment for cancer and then again subsequent to the subject having undergone therapeutic treatment for cancer, wherein a difference in the glycosyltransferase gene expression levels from before treatment to after treatment is indicative of the efficacy of the therapeutic treatment.

According to a further aspect of the invention there is provided a method of selection of a treatment regime for a subject suspected of having, or known to have cancer comprising performing the method of the invention on a sample from the subject and then selection of the appropriate treatment regime for the cancer type or subtype identified in the subject.

The method of selection may be performed prior to a subject undergoing therapeutic treatment for cancer and then again subsequent to the subject having undergone therapeutic treatment for cancer, wherein a difference in the glycosyltransferase gene expression levels from before treatment to after treatment is indicative of the efficacy of the therapeutic treatment.

According to a further aspect of the invention there is provided a method of treatment of a subject suspected of having, or known to have cancer, the method comprising:
(a) performing the method of the invention on a sample from the subject;
(b) identifying the subject's cancer type and optionally the subject's cancer subtype by the method of the invention;
(c) based on the results of step (b), determining whether to initiate treatment, modify the treatment regimen, or discontinue treatment of the subject; and
(d) optionally, initiating treatment, modifying the treatment regimen or discontinuing treatment.

The method of treatment may be performed prior to the subject undergoing therapeutic treatment for cancer and then again subsequent to the subject having undergone therapeutic treatment for cancer, wherein a difference in the glycosyltransferase gene expression profile of the subject from before treatment to after treatment is indicative of the efficacy of the therapeutic treatment.

According to a further aspect of the invention, there is provided a kit according to the invention for use in a method of treating cancer or selection of a treatment regime for a subject comprising the steps of:
(a) performing the method of the invention on a sample from the subject;
(b) identifying the subject's cancer type and optionally classification of the subject's cancer subtype by the method of the invention;
(c) based on the results of step (b), determining whether to initiate treatment, modify the treatment regimen, or discontinue treatment of the subject; and
(d) optionally, initiating treatment, modifying the treatment regimen or discontinuing treatment.

According to a further aspect of the invention there is provided glycosyltransferase gene polynucleotide sequences selected from the group consisting of the glycosyltransferase genes listed in Table 1 or fragments thereof for use with the method of the invention.

### BRIEF DESCRIPTION OF THE DRAWINGS

FIG. 1 shows the segregation of six TOGA cancer types using unsupervised clustering of the expression profile of 210 glycosyltransferase genes. Three-dimensional (3D) plot shows separation of six investigated cancer types (i.e. breast: breast invasive carcinoma [BRCA, n=531], ovarian: ovarian serous cystadenocarcinoma [OV, n=546], colon: colon adenocarcinoma [COAD, n=153], lung: lung squamous cell carcinoma [LUSC, n=154], brain: glioblastoma multiforme [GBM, n=400] and kidney: kidney renal clear cell carcinoma [KIRC, n=72]);

FIG. 2 shows the flow chart of supervised classification/class prediction of six TOGA cancer types along with the validation steps. The performance of the PAM classifier was examined using 10-fold cross validation, internal and external tests. For the purpose of classifier error estimation in the assignment of samples to the right cancer types, a 10-fold cross validation was carried out. Furthermore, for the purpose of internal validation, the glycosyltranseferases' expression dataset was randomly split hundred times into training (70%) and test (30%) sets. Training sets were used to build a model/classifier, which were then applied to the testing sets. Finally, the median values were used to assign each sample to a specific cancer type. Since training algorithms look for patterns in the training dataset, a classifier that relies on these spurious patterns will have higher accuracy on the training examples than it will on the whole population. Therefore, it is absolutely vital to evaluate the performance of a classifier on an independent test set. For this purpose, TOGA data has been used as training set for an external (independent) test that examine 293 breast cancer samples existing in GPL1390 platform of GSE20624 (Anders et al., 2011). GSE20624 (GPL1390) data is not included in TOGA while it uses the same microarray platform with TOGA datasets, however, only 177 glycosyltransferase are common between training (TOGA based) and this dataset;

FIG. 3 shows the evaluation of the PAM model/classifier made by the expression of 210 glycosyltransferase genes in six TOGA cancer types. A) 10-fold Cross Validation. Table summarize the result of 10-fold cross validation, which estimate accuracy of the model/classifier when an independent data set is used for tumour type prediction. The plot below the table shows the probability of each sample to assign to the right cancer type in each cancer type. Threshold plots were used to decide on the number of genes in training set, which give the less error rate. In this study the threshold was set to zero (t=0), which produces the least overall error rate in 10-fold cross validation and also to prevent the bias result of internal validation. B) Internal Validation. Circular heatmap representation of the results of 100 internal tests. Percentage values in the middle of heatmaps show the fraction of samples, which assign to the right tumour type with posterior probability 0.95. Value on the sidebar represents the median of posterior probability, which assigns each sample to a specific cancer type. The accuracy measures derived from a confusion matrix, the area under the receiver operating characteristic (ROC) curve and its confidence interval (CI) for internal test (table below), clearly shows the potential of gene expression profiling of glycosyltransferase in tumour type identification/separation with high accuracy, sensitivity and specificity for all investigated cancer types. C) External Validation. Performance evaluation of the model/classifier on an independent test set (GSE20624, non-TOGA). TOGA data set has been used as training for an external (independent) test that examine 293 breast cancer samples existing in GPL1390 platform of GSE20624 (Anders et al. 2011). GSE20624 (GPL1390) data is not included in TOGA while it uses the same microarray platform with TOGA datasets, however, only 177 glycosyltransferase are common between training (TOGA based) and this dataset;

FIG. 4 shows the class discovery of five breast cancer subtypes by gene expression profiling of 210 glycosyltransferase genes. (A) Consensus clustering matrix of 467 breast cancer (breast invasive carcinoma) samples with survival and PAM50 subtyping information in TOGA for k=3 to k=6 using k-means clustering method. (B) Consensus clustering CDF for k=2 to k=10 (the arrow shows the movement direction of CDF curve with increasing the number of clusters from 2 to 10). (C) SigClust p-values for all pairwise comparisons of five clusters/groups. (D) Log rank test p-values investigating the relation of number of subtypes (k=2 to k=10) with survival information. G1: group 1 (n=97), G2: group 2 (n=91), G3: group 3 (n=93), G4: group 4 (n=103), G5: group 5 (n=83);

FIG. 5 shows the clinical data and somatic mutation patterns along with survival curves comparing five breast cancer mRNA-expression subtypes (G1-G5) discovered by profiling the expression of 210 glycosyltransferase genes. Tumour samples with clinical data and somatic mutation information (n=467) in TOGA Agilent Microarray dataset for breast (breast invasive carcinoma) are grouped in five subtypes (G1-G5) based on their glycosyltransferase expression: Group 1 (G1, n=97), Group 2 (G2, n=91), Group 3 (G3, n=93), Group 4 (G4, n=103) and Group 5 (G5, n=83) (sample grouping based on the TOGA 2012 study is illustrated with a separate coloured side bar, Luminal A: blue, Luminal B: light blue, basal-like: red and HER2-enriched: pink). From left to right columns show clinical data (top) and frequencies in percentage (bottom) within samples for HER2, PR and ER (positive: white, negative: black). Somatic mutation patterns and frequencies for significantly mutated genes (wild type: white, truncation mutation: black and missense mutation: grey). Left plot illustrate the result of survival analysis of TOGA breast cancer samples with respect to the identified subtypes;

FIG. 6 shows the class discovery of six ovarian cancer subtypes by gene expression profiling of 210 glycosyltransferase genes. (A) Consensus clustering matrix of 529 ovarian cancer (ovarian serous cystadenocarcinoma) samples with survival information in TCGA for k=2 to k=8 using k-means clustering method. (B) Consensus clustering CDF for k=2 to k=10 (the arrow shows the movement direction of CDF curve with increasing the number of clusters from 2 to 10). (C) SigClust p-values for all pairwise comparisons of six clusters/groups. (D) Log rank test p-values investigating the relation of number of subtypes (k=2 to k=10) with survival information. G1: group 1 (n=59), G2: group 2 (n=97), G3: group 3 (n=107), G4: group 4 (n=78), G5: group 5 (n=79), G6: group 6 (n=109);

FIG. 7 shows the class discovery of seven brain cancer subtypes by gene expression profiling of 210 glycosyltransferase genes. (A) Consensus clustering matrix of 399 brain cancer (glioblastoma multiforme) samples with survival information in TCGA for k=3 to k=9 using k-means clustering method. (B) Consensus clustering CDF for k=2 to k=10 (the arrow shows the movement direction of CDF curve with increasing the number of clusters from 2 to 10). (C) SigClust p-values for all pairwise comparisons of seven clusters/groups. (D) Log rank test p-values investigating the relation of number of subtypes (k=2 to k=10) with survival information. G1: group 1 (n=48), G2: group 2 (n=33), G3: group 3 (n=73), G4: group 4 (n=33), G5: group 5 (n=93), G6: group 6 (n=34), G7: group 7 (n=85); and

FIG. 8 shows the survival analysis of ovarian and brain cancers with respect to the discovered subtypes using gene expression profile of 210 glycosyltransferase genes. Kaplan-Meier survival plots and p-values of the log rank test analysis according to six and seven subtypes for ovarian (high-grade serous ovarian carcinoma) (A) and brain (glioblastoma multiforme) (B) cancers.

### DETAILED DESCRIPTION OF THE INVENTION

This invention relates to a method of identification of cancer types in a subject comprising the use of supervised algorithms that have been trained to evaluate quantified glycosyltransferase gene expression in a sample from the subject. In addition, the method relates to a method of cancer subtype classification and/or identification of a cancer subtype within a specific cancer type comprising the use of unsupervised and supervised algorithms that have been trained to evaluate quantified glycosyltransferase gene expression. The invention further relates to a kit comprising glycosyltransferase gene capture probes or primers for use with the method, and a method of treatment of cancer comprising the use of the method or kit of the invention to select an appropriate treatment regime for a subject and/or to monitor the response of the subject to the cancer treatment.

The term “cancer” has come to describe complex malignant diseases that may not share the same causative agents, aetiology or molecular profiles. The complex nature of a solid tumour approaches that of normal tissues and has been compared to that of an organ with the discovery of the family of cell types making up its microenvironment. In an update to the well cited hallmarks of cancer Hanahan and Weinberg summed up the current understanding of the different roles tumour cells play in the progression of the disease (Hanahan and Weinberg 2011b). Tumour cells have an altered cellular biochemistry that facilitates evasion of growth suppressors and apoptosis as well as acquire invasive, angiogenic and metastatic properties. More pointedly it is unclear to what extent the degree of specific glycosylation mechanisms interface with the pathways. Neoplastic cells must first invade surrounding cells before they can metastasize. Cancer related oligosaccharide changes have been implicated with the invasive properties of cancer cells (Dall'Olio, 1996b). The structure of complex oligosaccharides are altered on normal cells that are on a neoplastic transformation path to malignancy has been documented over the last few decades. These post translational modifications (PTMs), orchestrated by the regulation of glycosyltransferase genes and the subsequent biochemical action of a glycosyltransferases, play key roles in the progression toward malignancy that is reliant on tumorigenesis, tumour progression and metastasis (Ohtsubo and Marth, 2006; Wang et al., 2009; Li et al., 2010; Li et al., 2013; Liu et al., 2013).

The inventors believe that the glycosyltransferase engineered changes of glycan structures covalently coupled to peptides, proteins and lipids are strongly implicated as signatures in malignant tumour typing and possibly subtyping. The inventors have therefore gone on to assess whether or not by monitoring the expression of glycosyltransferase genes on a genomic scale that this subset of genes provides an accurate means of cancer segregation and further identification of cancer type. Surprisingly, the inventors have shown that using the expression profiles of glycosyltransferase genes not only can different cancer types be identified from an unknown test sample, but furthermore, it is possible to classify cancer subtypes within a specific cancer type and optionally to predict a likely outcome for the subject having a particular cancer subtype.

As used herein, the term “gene” refers to a unit of inheritance, including the protein coding and noncoding transcribed regions, upstream and downstream regulatory regions, transcribed regions, and all variants of the mature transcript.

As used herein the term “cDNA” means synthetic DNA synthesized from a messenger RNA (rnRNA) template in a reaction catalysed by a reverse transcriptase enzyme.

As used herein, the terms “mRNA” and “mRNA transcript” are used interchangeably and mean an RNA molecule transcribed from the DNA of a gene.

As used herein, the phrase “clustering algorithm” means a bioinformatic mathematical process or set of rules to be followed in calculations or other problem-solving operations, especially by a computer for partitioning a given data set into groups based on specified features so that the data points within a group are more similar to each other than the points in different groups.

“Centroid” as used herein means the average gene expression for each gene in each cancer class (i.e. each cancer type), divided by the within-class standard deviation for that gene.

“Reference glycosyltransferase gene expression centroids” as used herein means a standardized glycosyltransferase gene expression centroid that has been computed for each cancer type by (i) calculating the average gene expression value for each glycosyltransferase gene selected from the group consisting of the glycosyltransferase genes listed in Table 1 in each cancer type, and (ii) dividing the average gene expression value by the within-class standard deviation for that gene.

“Nearest centroid classification” means (i) obtaining the glycosyltransferase gene expression profile of a test sample, (ii) comparing it to each reference glycosyltransferase gene expression centroid, and (iii) identifying the cancer type of the test sample as the cancer type of the centroid that the test sample is closest to, in squared distance.

“Consensus clustering” means a method of providing quantitative evidence for determining the number and membership of possible clusters within a dataset. The Consensus Clustering method involves subsampling from a set of items, such as microarrays, and determines clusterings of specied cluster counts (k). Then, pairwise consensus values, the proportion that two items occupied the same cluster out of the number of times they occurred in the same subsample, are calculated and stored in a symmetrical consensus matrix for each k (Monti et al, 2006).

As used herein, the term “oligonucleotide” means a short single-stranded nucleic-acid chain (either as an oligodeoxynucleotide or oligoribonucleotide). As used herein the term “polynucleotide” means a linear polymer whose molecule is composed of many nucleotide units.

As used herein, the term “cancer type” means the type of cancer as determined by the type of tissue in which the cancer originates (histological type) or by primary site, or the location in the body where the cancer first developed or the kind of cell from which it is derived, as well the appearance of the cancer cells.

As used herein, the term “cancer subtype” means a secondary classification of the cancer type, falling within the cancer type. It may be referred to as a molecular classification of cancer. In particular, the cancer subtype may be associated with molecular alterations, cancer survival, distinct clinical and pathological characteristics, specific gene expression signatures, and deregulated signalling pathways.

Glycans that mediate crucial pathophysiological events taking place at various stages of tumour progression are considered to be one of the key drivers of cancer biology. The applicants have assessed whether, since changes in specific glycan structures are essential for tumourigenesis, tumour progression and metastasis, identifying differences in the expression patterns of their glycosyltransferase genes might be a descriptive approach for understanding cancer complexity. Glycosyltransferase genes regulate the tissue-specific biosynthesis of glycan structures resulting in structural variations of the glycans.

Interestingly, the applicants have determined that the glycosyltransferase gene expression profile indeed can be used not only to identify the specific cancer types of a test or blind sample, but can also be used to classify or identify the test cancer into a subtype, including linking the subtype a particular survival prognosis.

The molecular techniques referenced herein, including RNA extraction, purification amplification, cDNA amplification, mRNA and cDNA labelling, primers and probes design, Northern and Southern blotting, microarray printing and methods, RT-PCR, RT-qPCR, and real time RT-PCR are all standard methods known to those skilled in the art. Many reference sources are available, including but not limited to: http://www.qiagen.com/resources/molecular-biology-methods/, Green and Sambrook, 2012, and others known to those skilled in the art.

The invention will be described by way of the following examples which are not to be construed as limiting in any way the scope of the invention.

### EXAMPLE 1

### Sic Cancers Represented in the Cancer Genome Atlas (TCGA)

1. Methods

For the purpose of this study, Agilent Microarray (Agilent 244K custom gene expression) data of 1893 samples from published TOGA (http://tcga-data.nci.nih.gov/tcga/) representing six cancer types including breast: breast invasive carcinoma [BRCA, n=531], ovary: ovarian serous cystadenocarcinoma [OV, n=578], brain: glioblastoma multiforme [GBM, n=403], kidney: kidney renal clear cell carcinoma [KIRC, n=72], colon: colon adenocarcinoma [COAD, n=154] and lung: lung squamous cell carcinoma [LUSC, n=155]) were combined, while normal and control samples were excluded.

A list of human glycosyltransferase genes was retrieved through filtering several publicly available databases such as Kyoto Encylopedia of Genes and Genomes database (KEGG/GENES) (http://www.genome.jp/kegg/genes.html), Carbohydrate-Active Enzymes database (CAZy) databases (http://www.cazy.org/), and literature search. KEGG/GENES is a pool of manually curated genes retrieved mainly from NCBI RefSeq (Pruitt et al., 2005; Kanehisa et al., 2006; Pruitt et al., 2007). Furthermore, CAZy provides an online and regularly updates access to family classification of CAZymes corresponding to proteins released in the daily releases of GenBank (ftp://ftp.ncbi.nih.gov/genbank/daily-nc) (Lombard et al., 2014). Table 1 contains a list of 210 glycosyltransferase gene symbols and their Entrez number.

The expression dataset of glycosyltransferases was built through combining the TCGA expression datasets of six investigated cancer types (i.e. breast, brain, colon, kidney, lung and ovarian) and further retrieving the expression of 210 glycosyltransferase genes from the combined dataset. The result of batch effect analysis clearly illustrated that no significant batch effects in the dataset (data not shown).

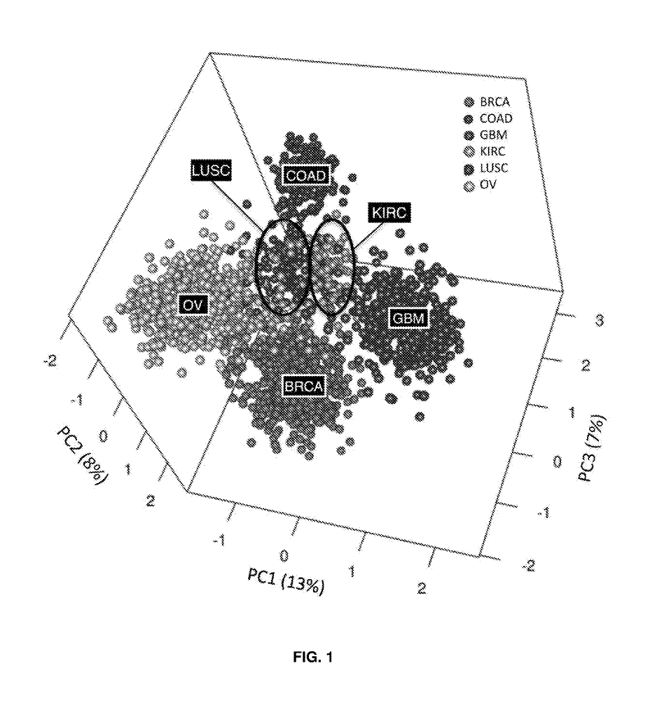

To illustrate the segregation of cancer types based on the expression of glycosyltransferase genes, principal component analysis (see FIG. 1) was performed using ‘psych’ package (Revelle, 2014) in R.

To evaluate the potential of glycosyltransferase expression profile in identifying cancer types, a model/classifier was build using ‘pamr’ package (Hastie et al., 2003) in R and further evaluate using 10-fold cross validation, internal and external tests (see FIG. S2). A two-by-two confusion matrix (also called a contingency table) was constructed representing the dispositions of the set of samples, see Fawcett (2006) for more information and equations. Furthermore, the ROC (Receiver Operating Characteristics) curve and the area under the ROC (AUC) (Bradley, 1997) were also investigated to evaluate the performance of the classifier using ‘rpROC’ package (Robin et al., 2011) in R. Furthermore, the performance of the classifier was evaluated using an independent test set. For this purpose, TCGA data set has been used as training set for an external (independent) test that examine 293 breast cancer samples existing in GPL1390 platform of GSE20624 (Anders et al., 2011).

2. Results and Discussion

The microarray expression data of 1893 samples were retrieved from The Cancer Genome Atlas (TCGA) project (http://tcga-data.nci.nih.gov/tcga/), representing six cancer types including breast: breast invasive carcinoma [BRCA, n=531], ovary: ovarian serous cystadenocarcinoma [OV, n=578], brain: glioblastoma multiforme [GBM, n=403], kidney: kidney renal clear cell carcinoma [KIRC, n=72], colon: colon adenocarcinoma [COAD, n=154] and lung: lung squamous cell carcinoma [LUSC, n=155]). The applicant further combined and filtered the data to derive at the expression of 210 glycosyltransferase genes.

Table 1 provides a list of 210 glycosyltransferase genes present in the dataset.

To assess the ability of glycosyltransferase expression profile in separating different cancer types Principal Component Analysis (PCA) (Hotelling, 1933) was performed in which the first three principal components with the highest variances (PC1, PC2 and PC3) explained 28% of variations between the cancer types assessed and were clearly shown to separate different cancer types (see FIG. 1).

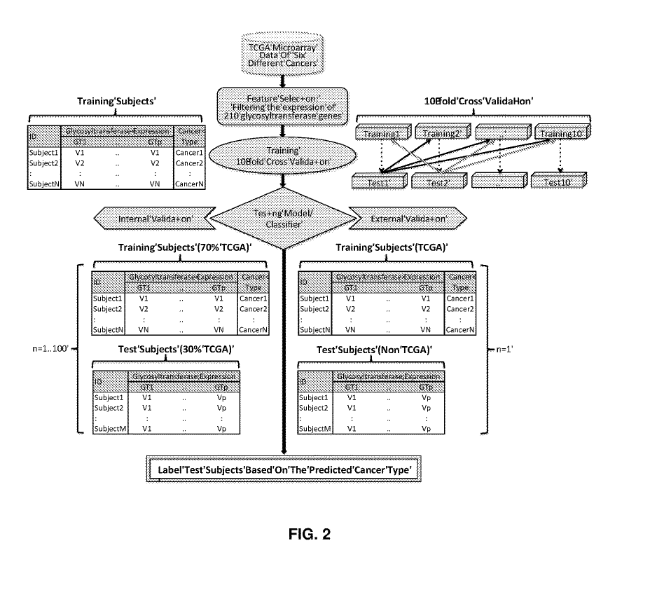

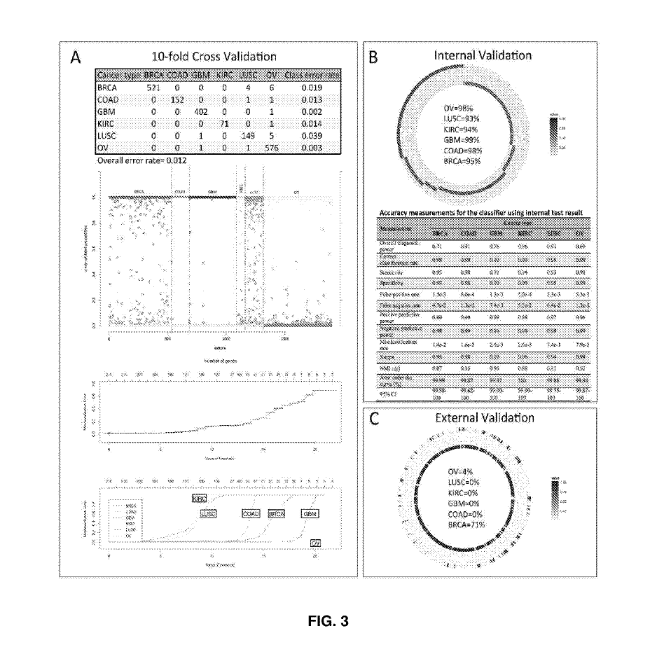

To evaluate the potential of glycosyltransferase expression profile in identifying cancer types, a model/classifier was build using ‘pamr’ package (see FIG. 2). For the purpose of error estimation in the assignment of samples to the right cancer types, a 10-fold cross validation technique was carried out using ‘pamr’ package (see FIG. 3A).

In addition, internal and independent/external tests were carried out to evaluate the performance of the pam classifier using the expression of glycosyltransferase genes. For the purpose of internal validation, the glycosyltranseferases' expression dataset was randomly split hundred times into training (70%) and test (30%) sets. Training sets were used to build the models/classifiers, which were then applied to the testing sets. Finally, the median values (centroids) were used to assign each sample to a specific cancer type. The result of this analysis was used for accuracy measurement calculation summarized in FIG. 3B. The accuracy measures derived from a confusion matrix, the area under the receiver operating characteristic (ROC) curve and its confidence interval (CI) for internal test (see FIG. 3B), clearly shows the potential of gene expression profiling of glycosyltransferase in tumour type prediction/identification with high accuracy (>98%), sensitivity (>93%) and specificity (>98%) for all investigated cancer types (see table in FIG. 3B), while at least 93% of test samples are assigned to the right cancer type in internal test (see circular heatmap in FIG. 3B).

Since training algorithms look for patterns in the training dataset, a classifier that relies on these spurious patterns will have higher accuracy on the training examples than it will on the whole population. Therefore, it is absolutely vital to evaluate the performance of a classifier on an independent test set. For this purpose, training sets of previous test (internal test) have been used for an external (independent) test that examine 293 breast cancer samples existing in GPL1390 platform of GSE20624 (Anders el al., 2011). GSE20624 (GPL1390) data is not included in TOGA while it uses the same microarray platform with TOGA datasets, however, only 177 glycosyltransferase are common between training (TOGA based) and this dataset. The result of this test was illustrated as a circular heatmap (see FIG. 3C).

We thus, determined estimations for tumour type separation of more than 93% and 71% (posterior probability 0.95) in internal and external tests, respectively (see circular heatmaps in FIG. 3B and C).

3. Conclusion

DNA microarray experiments generate large datasets with simultaneous monitoring of thousands of gene expressions. Accurate identification of different tumour types and subtypes from the unknown samples in such high-dimensional data is difficult but often crucial for successful diagnosis and treatment.

The results of this study show that the expression profiles of glycosyltransferase genes are significantly altered between different cancer types and these differences are enough to identify cancer type and optionally subtypes in a subject. Therefore this study offers a novel and improved alternative method compared to current cancer diagnosis techniques.

### EXAMPLE 2

### Cancer Subtyping

1. Methods

The inventors have assessed the use of glycosyltransferase gene expression profiles in subtype classification and identification. Samples from three different cancers were obtained: breast, ovarian and brain cancer, and these were analysed for the provision of a quantitative evidence for the prediction of a number of possible subtypes within the each cancer dataset by consensus clustering plus class discovery technique (Wilkerson and Hayes, 2010) using ConsensusClusterPlus' package (Monti et al. 2003) in R. The consensus Cumulative Distribution Function (CDF) is a graphical representation to illustrate at what number of clusters, the CDF reaches an approximate maximum and at which k (number of groups) there is no significant increase in CDF curve, respectively. Furthermore, to group samples into subtypes based on the expression of glycosyltransferase genes, a k-means clustering was performed using ‘cluster’ package (Meechler et al., 2012) in R. In addition, cluster significance was evaluated using ‘SigClust’ package (Huard et al., 2012) in R. To investigate whether the identified groups (using k-means clustering), specific to each of the cancer types may represent clinically distinct subgroups of patients; univariate survival analyses (comparing subtypes, k=2 to k=10, with respect to the overall survival) was performed using ‘survival’ package (Therneau, 2013) in R.

In case of breast cancer, previously identified normal-like (TCGA 2012), metastatic samples and the samples with missing survival information in the corresponding patient were excluded. Furthermore, clinical and somatic mutation patterns along with survival curves were compared within the context of the five discovered subtypes by the expression of glycosyltransferase genes.

In terms of ovarian and brain cancers, the samples with missing survival information in the corresponding patient were excluded.

2. Results and Discussion

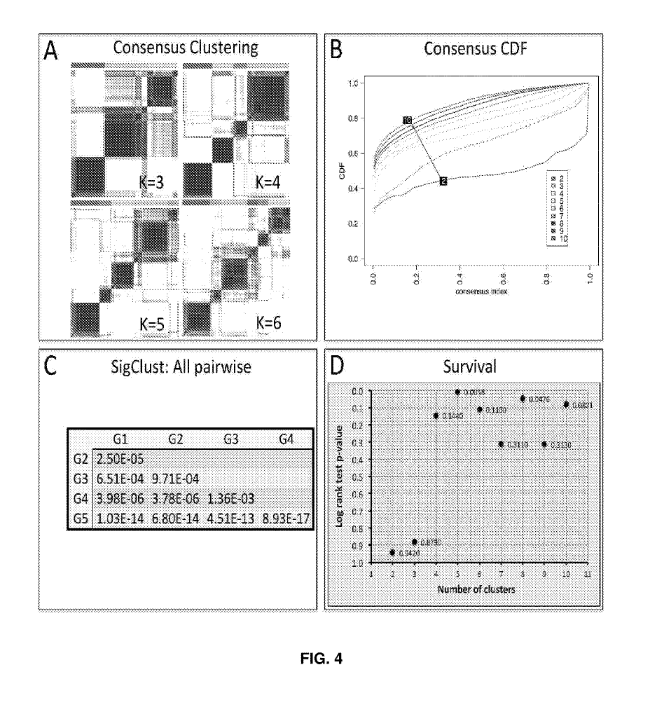

In addressing the ability of glycosyltransferase genes in each of the cancer types to provide subtype classification, the inventors performed consensus average linkage clustering that allows a quantitative and visual assessment of the estimated number of unsupervised subtypes in a particular cancer (Prat et al., 2013a). The results indicate that clustering stability increases from k=2 to k=10(see FIGS. 4, 6 and 7) and it may explain the heterogeneity observed in each cancer type, while further identifying molecular subtypes within or in addition to previously identified classes.

### Breast Cancer Subtypes

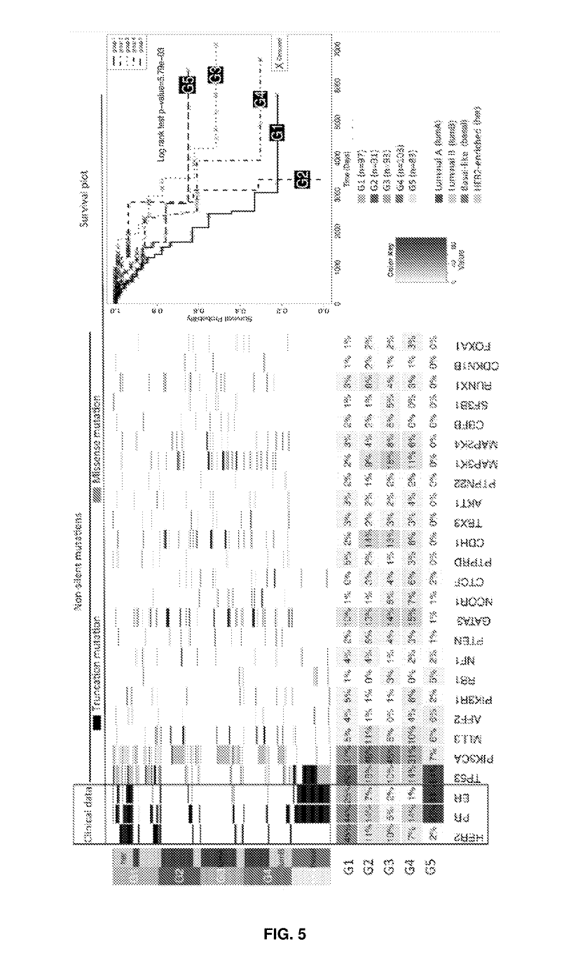

The inventor's results pertaining to the prognostic evaluation of breast cancer subtypes using the expression profile of glycosyltransferase genes indicate that survival curves significantly differ between subtypes when breast cancer samples are divided into five groups (see FIG. 4D and 5). According to the study carried out by Perou et al. (2000), Sorlie et al. (2001) and several others, the existence of four major breast cancer subtypes (Luminal-type A, Luminal-type B, Basal-like and HER2-enriched) has become a consensus in breast cancer classification. Furthermore, a study carried out by The Cancer Genome Atlas Network, TOGA (2012), provided key insights into these four previously defined breast cancer subtypes and explains significant molecular heterogeneity in each subtypes.

In line with these findings, the inventor's results show the most significant difference between breast cancer subtypes when data is divided into five groups (Log rank test p-value=5.79e-03). Furthermore, clinical and somatic mutation patterns along with survival curves were compared within the context of the five detected subtypes (G1-G5) using the expression profile of glycosyltransferase genes (see FIG. 5). The result of this analysis was shown that there is a significant overlap between G1 and HER2-enriched, and G5 and basal-like subtypes, while luminal (A and B) are mainly distributed between other three subtypes (G2, G3 and G4). Clinical patterns were significantly more conserved within G1 (HER2+: 45%, PR−: 44% and ER−: 25%, considered as HER2-enriched) and G5 (HER2+: 2%, PR−: 93% and ER−: 84%, considered as basal-like) in compare to other three subtypes. The samples in G1 and G5 subtypes are mostly mutated for TP53 (62% and 84% respectively), while G1 and G5 are differently mutated for PIK3CA (37% and 7% respectively). TP53 mutations are significantly enriched in HER2+or ER− tumours, while a high frequency of PIK3CA mutations was reported in HER+ tumours (TCGA, 2012). Therefore, the investigation of mutation frequency of TP53 and PIK3CA in G1 (62% and 37% respectively) and G5 (84% and 7% respectively) are extra evidence beside the clinical data confirming these two identified subtypes using glycosyltransferase expression profile might be Her2-enriched and basal-like subtypes and they are different from luminal subtypes. Furthermore, GATA3 mutations rarely found in G1 (1%) compare to other groups (10%, 13%, 14% and 15% for G1 to G4 respectively) in this study. Since GATA3 mutations are only observed in luminal subtypes or ER+ tumours (TOGA, 2012), which might be extra support for G5 to be considered as basal-like subtype that glycosyltransferase expression profile clearly identified and separated this subtype from other subtypes.

Among luminal subtypes (G2, G3 and G4), G4 has the lowest rate of HER2+and ER− (HER2+: 7% and ER-: 1%), while G2 and G3 have the highest ER- (7%) and lowest PR− (5%) respectively. The samples in G4 (considered as luminal/ER+/HER2−) subtype are moderately mutated for TP53 (14%) and PIK3CA (31%), followed by GATA3 (15%), MAP3K1 (11%) and MLL3 (10%). Estrogen is the prime growth regulator of ER-positive breast cancer and GATA3 is an ER-regulated gene and play key roles in ER-mediated target gene regulation and it is important for the genomic action of the ER (Ma et al. 2013). Mutations in this gene could potentially mark disease that is sensitive to endocrine therapy (Ellis et al., 2012a).

Furthermore, the rate of GATA3 mutation in luminal subtypes (G2: 13%, G3: 14% and G4: 15%) is in line with the result of the other studies, which reported the GATA3 mutation at −10% frequency investigating a significant number of luminal tumours (Banerji et al., 2012; Ellis et al., 2012a; Stephens et al., 2012; TCGA, 2012). Another feature of the somatic mutated gene list in ER+/HER2− breast cancer is the presence of additional genes previously implicated in leukemia and myelodysplasia such as MLL3 and RUNX1 (Ellis and Perou, 2013). MLL family members encode histone trimethyltransferases—considered positive global regulators of gene transcription, with functions that include regulation of estrogen receptor gene expression (Won Jeong et al. 2012). Theoretically MLL mutant tumors, as well as others with mutations in genes that are epigenetic regulators of gene expression, might be sensitive to the burgeoning class of drugs that target post-translational histone modifications (Huang et al., 2011).

G2 and G3 subtypes are significantly enriched for luminal A mRNA subtype. The samples in G3 subtype (considered as luminal/ER+/HER2+) are rarely mutated for TP53 (10%) but mostly mutated for PIK3CA (45%), followed by GATA3 (14%), CDH1 (13%) and MAP3K1 (18%), while the samples in G2 subtype (considered as luminal/ER-/HER2+) are mostly mutated for TP53 and PIK3CA (18% and 48% respectively) compare to other luminal subtypes followed by MLL3 (11%), GATA3 (13%) and CDH1 (14%). CDH1 mutations are correlated with a significant alteration in HER2 (Ross et al. 2013), while MAP3K1 mutations often coexist with PI3KCA mutations in luminal A and shown to be linked with low proliferative index before and after estrogen deprivation therapy (Ellis et al., 2012a). After TP53, mutation rate of MAP3K1 significantly vary between G2 and G3 subtypes (9% and 18% respectively). MAP3K1 mutation is a high frequency driver mutation for Luminal A disease and therefore also a likely causal molecular event (Ellis and Perou, 2013), which may lead to the molecular subtypes in luminal disease.

In conclusion, investigating the expression profile of glycosyltransferase genes provide a clear distinction between breast cancer subtypes (Luminal A/B, Basal-like and HER2− enriched) and may provide key insights into molecular heterogeneity of luminal subtypes considering subtype-associated gene mutations.

### Ovarian and Brain Cancer Subtypes

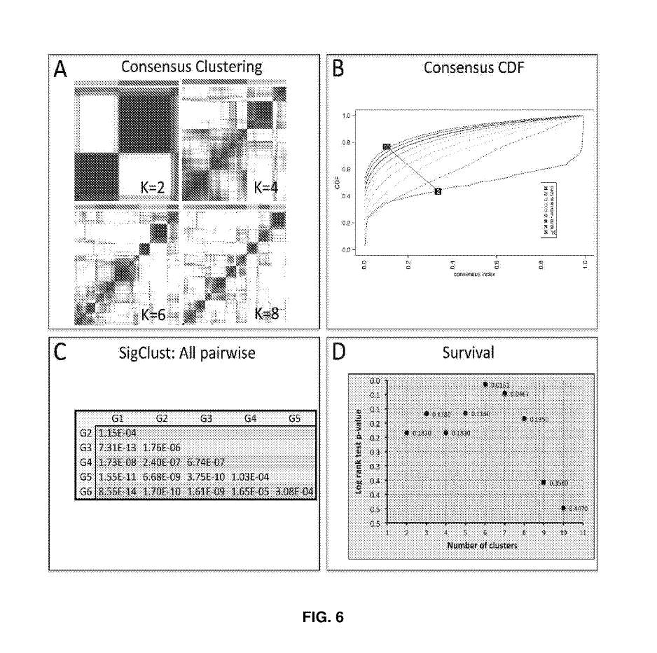

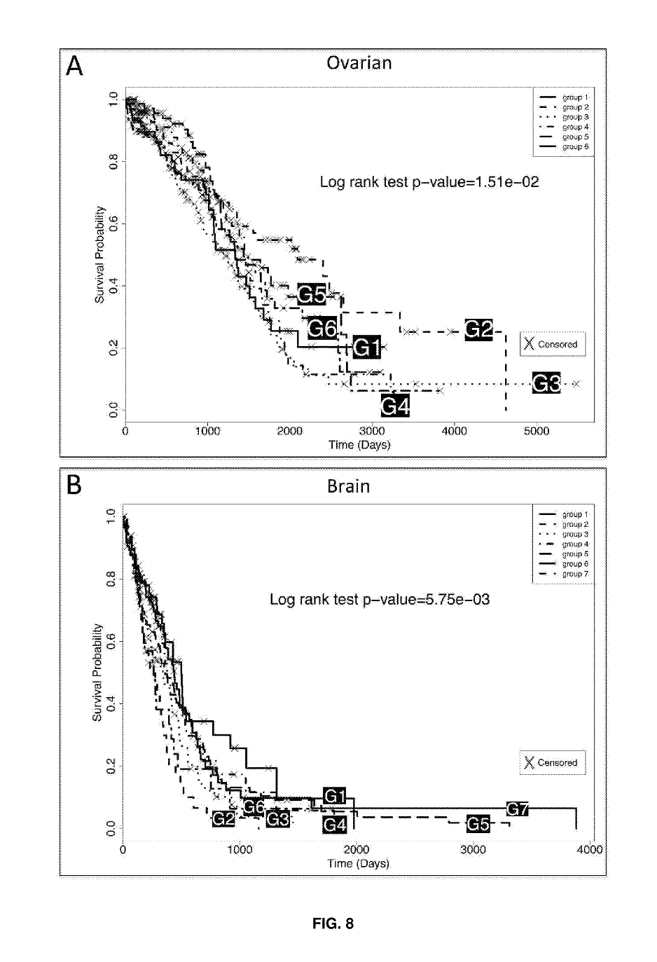

To investigate whether the identified groups (using the same methods that have been explained for breast cancer), specific to ovarian and brain cancer types may represent clinically distinct subgroups of patients, the inventor's performed a univariate survival analyses (comparing subtypes with respect to the overall survival). Survival curves were found to significantly differ between different cancer subtypes when ovarian and brain cancers were divided (see FIG. 8). In terms of ovarian cancer, the most significant difference between survival curves of subtypes was achieved when data divided into six subtypes (Log rank test p-value=1.51e-02) (see FIGS. 6 and 8) despite, four, five and six molecular subtypes having been previously reported by The Cancer Genome Atlas Network, TOGA (2011), Tan et al. (2013) and Tothill et al. 2008), respectively.

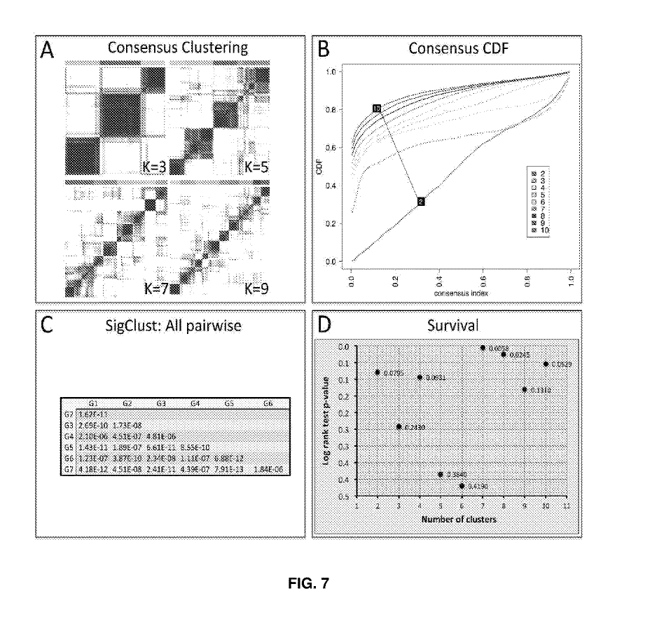

Furthermore, the inventors showed the presence of seven subtypes for brain (Log-rank test p-value=5.75e-03) (see FIGS. 7 and 8), while the number of brain subtypes previously reported by Verhaak et al., (2010) and Brennan et al. 2013) is four and five, respectively.

### Conclusion

In conclusion, the heterogeneity presented between discovered breast cancer subtypes and homogeneity displayed within each subtype in terms of clinical and mutation patterns may confirm the potential of using the expression profile of glycosyltransferase genes in cancer subtyping and outcome prediction. Furthermore, the significance of the inventor's findings lies not only in finding previously reported cancer subtypes but also their significant correlation with clinical outcome (and mutation patterns in case of breast cancer) provides a supporting evidence for the potential use of glycosyltransferase gene expression profiles in cancer class subtyping.

4. Final conclusion

Personalized medicine in the context of cancer, is closely tied with the concept of accurately identifying cancer types and furthermore subtypes of a specific cancer in a subject for accurate diagnosis, predict an outcome and hence advise on better treatment.

Although glycan alteration may be indicative of the specificity of glycan epitopes to their associated cancer group, the fundamental and precise structures of glycans are complicated and largely not understood. Thus, in the absence of this information, the use of glycosyltransferase gene expression profiles is an compelling alternative with the diagnosis potential, with in-depth understanding of the complexity of different cancer types at the translational level.

The altered expression of glycosyltransferase genes whose expressions are significantly related to cancer patient survival represent compelling evidence for the role of these genes in cancer type identification and subtyping. Therefore, glycosyltransferase genes represent useful targets for use in clinical assays, resulting in the development of improved early diagnosis.

REFERENCES

[1]. D. Hanahan, R. A. Weinberg, Cell 144, (2011a)

[2]. F. Dall'Olio, Clin Mol Pathol. 49, (1996a)

[3]. K. Ohtsubo, J. D. Marth, Cell 126, (2006)

[4]. W. Wang, et al., Proc Natl Acad Sci USA 106, (2009)

[5]. M. Li, et al., J Biosci 35, (2010)

[6]. S. Li, et al., PLoS One 8, (2013)

[7]. L. Liu, et al., PLoS One 8, (2013)

[8]. C. K. Anders, et al., Journal of Clinical Oncology 29, (2011)

[9]. J. T. Leek, et al., Bioinformatics 28, (2012)

[10]. D. Hanahan, R. A. Weinberg, cell 144, (2011b)

[11]. F. Dall'Olio, Clinical molecular pathology 49, (1996b)

[12]. K. D. Pruitt, et al., Nucleic Acids Res 33, (2005)

[13]. M. Kanehisa, et al., Nucleic Acids Res 34, (2006)

[14]. K. D. Pruitt, et al., Nucleic Acids Res 35, (2007)

[15]. V. Lombard, et al., Nucleic Acids Res 42, (2014)

[16]. W. Revelle, R package (2014)

[17]. M. R. Green and J. Sambrook, Molecular Cloning: A Laboratory Manual, (2012)

[18]. T. Hastie, et al., R package (2003)

[19]. T. Fawcett, Pattern Recognition Letters 27, (2006)

[20]. A. P. Bradley, Pattern recognition 30, (1997)

[21]. X. Robin, et al., BMC bioinformatics 12, (2011)

[22]. H. Hotelling, Journal of educational psychology 24, (1933)

[23]. A. Prat, et al., Scientific Reports 3, (2013a)

[24]. M. D. Wilkerson, D. N. Hayes, Bioinformatics 26, (2010)

[25]. M. Maechler, et al., R package (2012)

[26]. S. Monti, et al., Machine learning 52, (2003)

[27]. H. Huang, et al., R package (2012)

[28]. T. Therneau, R package (2013)

[29]. TCGA, Nature 490, (2012)

[30]. A. Prat, et al., Sci Rep 3, (2013b)

[31]. C. M. Perou, et al., Nature 406, (2000)

[32]. T. Sorlie, et al., Proc Natl Acad Sci USA 98, (2001)

[33]. C. X. Ma, et al., Breast cancer research and treatment 139, (2013)

[34]. M. J. Ellis, et al., Nature 486, (2012a)

[35]. M. J. Ellis, et al., Nature 486, (2012b)

[36]. S. Banerji, et al., Nature 486, (2012)

[37]. P. J. Stephens, et al., Nature 486, (2012)

[38]. M. J. Ellis, C. M. Perou, Cancer discovery 3, (2013)

[39]. K. Won Jeong, et al., Molecular Endocrinology 26, (2012)

[40]. Y. Huang, et al., Breast Cancer Research : BCR 13, (2011)

[41]. J. S. Ross, et al., Clinical Cancer Research 19, (2013)

[42]. TCGA, Nature 474, (2011)

[43]. T. Z. Tan, et al., EMBO molecular medicine 5, (2013)

[44]. R. W. Tothill, et al., Clinical Cancer Research 14, (2008)

[45]. R. Verhaak, et al., Cancer Cell 17, (2010)

[46]. C. W. Brennan, et al., Cell 155, (2013)

1-34. (canceled)

35. A method of identification of cancer type from the group consisting of six cancer types selected from breast cancer, high-grade serous ovarian carcinoma, glioblastoma multiforme, colon adenocarcinoma, kidney renal clear cell carcinoma, brain lower grade glioma and lung adenocarcinoma in a sample from a test subject comprising the steps of:
(a) obtaining a glycosyltransferase gene expression profile from the group consisting of 210 glycosyltransferase genes listed in Table 1 from the sample from the test subject, comprising the steps of:
A. purifying total RNA from the sample;
B. converting the RNA into cDNA by reverse transcription and labelling the cDNA with a fluorescent dye or amplifying mRNA from the RNA and labelling the mRNA with a fluorescent dye;
C. contacting a microarray slide printed with target oligonucleotide probes specific for the glycosyltransferase genes or a gene fragment of each selected from the group consisting of the glycosyltransferase genes listed in Table 1 with the fluorescently labelled cDNA or mRNA;
D. hybridizing the fluorescently labelled cDNA or mRNA to the target oligonucleotide probes; and
E. measuring the intensity of fluorescence of cDNA or mRNA bound to their target oligonucleotide probes on the microarray slide, whereby the intensity of fluorescence at each target corresponds to the relative abundance of mRNA transcript for each subject glycosyltransferase gene which is the test subject sample glycosyltransferase gene expression profile; or
A1. purifying total RNA from the sample of the subject;
B1. converting the RNA into cDNA by reverse transcription and labelling the cDNA with a fluorescent dye or a chromogenic substrate, or amplifying mRNA from the RNA and labelling the mRNA with a fluorescent dye or a chromogenic substrate;
C1. contacting a dot blot printed with target oligonucleotide probes specific for the 210 glycosyltransferase genes or a gene fragment of each selected from the group consisting of the glycosyltransferase genes listed in Table 1 with the labelled cDNA or mRNA;
D1. hybridizing the labelled cDNA or mRNA to the target oligonucleotide probes; and
E1. measuring the intensity of the hybridized labelled cDNA or mRNA bound to their target oligonucleotide probes on the dot blot, whereby the intensity of the label at each target corresponds to the relative abundance of mRNA transcript for each gene, that which is, the subject sample glycosyltransferase gene expression profile; or
A2. purifying total RNA from the sample;
B2. amplifying cDNA from the RNA by RT-qPCR with specific primers for the glycosyltransferase genes or a gene fragment of each selected from the group consisting of the glycosyltransferase genes listed in Table 1;
C2. measuring the amplified cDNA; and
D2. assessing the relative abundance of the amplified cDNA of each of the genes, whereby the abundance of the amplified cDNA corresponds to the relative abundance of mRNA transcript for each gene which is the glycosyltransferase gene expression profile;
(b) calculating the distance of the gene expression profile of the sample to a reference set of glycosyltransferase gene expression centroids from the six cancer types; and
(c) identifying the cancer type of the sample, wherein the cancer type is identified as the cancer type corresponding to the nearest reference set glycosyltransferase gene centroid of the six cancer types glycosyltransferase gene expression centroids compared with the glycosyltransferase gene expression profile from the sample.

36. A method of identification of cancer type from the group consisting of six cancer types selected from breast cancer, high-grade serous ovarian carcinoma, glioblastoma multiforme, colon adenocarcinoma, kidney renal clear cell carcinoma, brain lower grade glioma and lung adenocarcinoma in a sample from a test subject comprising the steps of:
(a) obtaining a glycosyltransferase gene expression profile for each of a training set of cancer samples from subjects with each of the six cancer types, wherein the glycosyltransferase genes are selected from the group consisting of the 210 glycosyltransferase genes listed in Table 1, comprising the steps of:
A. purifying total RNA from the samples;
B. converting the RNA into cDNA by reverse transcription and labelling the cDNA with a fluorescent dye or a chromogenic substrate, or amplifying mRNA from the RNA and labelling the mRNA with a fluorescent dye or a chromogenic substrate;
C. contacting a microarray slide printed with target oligonucleotide probes specific for the 210 glycosyltransferase genes or a gene fragment of each with the labelled cDNA or mRNA;
D. hybridizing the labelled cDNA or mRNA to the target oligonucleotide probes; and
E. measuring the intensity of the hybridized labelled cDNA or mRNA bound to their target oligonucleotide probes on the microarray slide, whereby the intensity of the label at each target corresponds to the relative abundance of mRNA transcript for each gene which is the training set glycosyltransferase gene expression profiles; or
A1. purifying total RNA from the sample(s);
B1. converting the RNA into cDNA by reverse transcription and labelling the cDNA with a fluorescent dye or a chromogenic substrate, or amplifying mRNA from the RNA and labelling the mRNA with a fluorescent dye or a chromogenic substrate;
C1. contacting a dot blot printed with target oligonucleotide probes specific for the 210 glycosyltransferase genes or a gene fragment of each with the labelled cDNA or mRNA;
D1. hybridizing the labelled cDNA or mRNA to the target oligonucleotide probes; and
E1. measuring the intensity of the hybridized labelled cDNA or mRNA bound to their target oligonucleotide probes on the dot blot, whereby the intensity of the label at each target corresponds to the relative abundance of mRNA transcript for each gene which is the glycosyltransferase gene expression profile; or
A2. purifying total RNA from the sample(s);
B2. amplifying cDNA from the RNA by RT-qPCR with specific primers for the glycosyltransferase genes or a gene fragment of each selected from the group consisting of the glycosyltransferase genes listed in Table 1;
C2. measuring the amplified cDNA; and
D2. assessing the relative abundance of the amplified cDNA of each of the genes, whereby the abundance of the amplified cDNA corresponds to the relative abundance of mRNA transcript for each gene which is the glycosyltransferase gene expression profile;
(b) using a supervised algorithm to construct gene expression centroids for each of the six cancer types in the training set;
(c) obtaining a glycosyltransferase gene expression profile from a set of glycosyltransferase genes selected from the group consisting of the 210 glycosyltransferase genes listed in Table 1 in a sample from the test subject comprising the steps of
A. purifying total RNA from the sample;
B. converting the RNA into cDNA by reverse transcription and labelling the cDNA with a fluorescent dye or a chromogenic substrate, or amplifying mRNA from the RNA and labelling the mRNA with a fluorescent dye or a chromogenic substrate;
C. contacting a microarray slide printed with target oligonucleotide probes specific for the 210 glycosyltransferase genes or a gene fragment of each with the labelled cDNA or mRNA;
D. hybridizing the labelled cDNA or mRNA to the target oligonucleotide probes; and
E. measuring the intensity of the hybridized labelled cDNA or mRNA bound to their target oligonucleotide probes on the microarray slide, whereby the intensity of the label at each target corresponds to the relative abundance of mRNA transcript for each gene which is the test subject sample glycosyltransferase gene expression profile; or
A1. purifying total RNA from the sample(s);
B1. converting the RNA into cDNA by reverse transcription and labelling the cDNA with a fluorescent dye or a chromogenic substrate, or amplifying mRNA from the RNA and labelling the mRNA with a fluorescent dye or a chromogenic substrate;
C1. contacting a dot blot printed with target oligonucleotide probes specific for the 210 glycosyltransferase genes or a gene fragment of each with the labelled cDNA or mRNA;
D1. hybridizing the labelled cDNA or mRNA to the target oligonucleotide probes; and
E1. measuring the intensity of the hybridized labelled cDNA or mRNA bound to their target oligonucleotide probes on the dot blot, whereby the intensity of the label at each target corresponds to the relative abundance of mRNA transcript for each gene which is the glycosyltransferase gene expression profile; or
A2. purifying total RNA from the sample(s);
B2. amplifying cDNA from the RNA by RT-qPCR with specific primers for the glycosyltransferase genes or a gene fragment of each selected from the group consisting of the glycosyltransferase genes listed in Table 1;
C2. measuring the amplified cDNA; and
D2. assessing the relative abundance of the amplified cDNA of each of the genes, whereby the abundance of the amplified cDNA corresponds to the relative abundance of mRNA transcript for each gene which is the glycosyltransferase gene expression profile;
(d) calculating the distance of the gene expression profile of the test subject sample to each of glycosyltransferase gene expression centroids from the training set of the six cancer types; and
(e) identifying the cancer type of the test subject sample wherein the cancer type is identified as that corresponding to the nearest training set glycosyltransferase gene centroid of the six cancer types compared with the glycosyltransferase gene expression profile from the test subject sample.

37. The method according to claim 35, wherein the method further comprises a step of classifying the subtype of the cancer in a test subject having one of the six cancer types, wherein the subtype classification comprises the steps of:
(a) obtaining a glycosyltransferase gene expression profile from a set of glycosyltransferase genes in a test sample from the test subject, wherein the glycosyltransferase genes are selected from the group consisting of the 210 glycosyltransferase genes listed in Table 1;
(b) providing a reference set of glycosyltransferase gene expression profiles from subtypes of the corresponding cancer type to that of the subject, wherein the glycosyltransferase genes are selected from the group consisting of the 210 glycosyltransferase genes listed in Table 1;
(c) performing unsupervised clustering analysis on the test subject glycosyltransferase gene expression profile and the reference set of glycosyltransferase gene expression profiles; and
(d) classifying the test subject subtype by grouping the test glycosyltransferase gene expression profile into one or more subtypes corresponding to the closest reference glycosyltransferase gene expression profile for a subtype.

38. The method according to claim 35, whereby the method further comprises a step of classifying the subtype of the cancer in a test subject having one of the six cancer types, wherein the subtype classification comprises the steps of:
(a) obtaining a glycosyltransferase gene expression profile from a set of glycosyltransferase genes in a sample from the test subject, wherein the glycosyltransferase genes are selected from the group consisting of the 210 glycosyltransferase genes listed in Table 1;
(b) using a supervised algorithm to construct a set of reference glycosyltransferase gene expression centroids from subtypes of the corresponding cancer type to that of the subject wherein the glycosyltransferase genes are selected from the group consisting of the 210 glycosyltransferase genes listed in Table 1;
(c) calculating the distance of the gene expression profile of the test sample to the reference glycosyltransferase gene expression centroids from the subtypes of the corresponding cancer to that of the subject; and
(d) classifying the cancer subtype of the test sample wherein the cancer subtype is classified as that corresponding to the nearest reference set glycosyltransferase gene centroid compared with the glycosyltransferase gene expression profile from the test sample.

39. The method according to claim 36, wherein the method further comprises a step of classifying the subtype of the cancer in a test subject having one of the six cancer types, wherein the subtype classification comprises the steps of:
(a) obtaining a glycosyltransferase gene expression profile for each of a training set of glycosyltransferase genes of cancer subtypes from subjects with the corresponding cancer type to that of the test subject, wherein the glycosyltransferase genes are selected from the group consisting of the glycosyltransferase genes listed in Table 1;
(b) using a supervised algorithm to construct gene expression centroids for each of the cancer subtypes in the training set;
(c) obtaining a glycosyltransferase gene expression profile from a set of glycosyltransferase genes in a sample from the test subject, wherein the glycosyltransferase genes are selected from the group consisting of the glycosyltransferase genes listed in Table 1;
(d) calculating the distance of the gene expression profile of the test sample to each of glycosyltransferase gene expression centroids constructed in step (b); and
(e) classifying the cancer subtype of the test sample wherein the cancer subtype is classified as that corresponding to the nearest training set glycosyltransferase gene centroid compared with the glycosyltransferase gene expression profile from the test sample.

40. The method according to claim 35 wherein the step of measuring the intensity of fluorescence of cDNA or mRNA bound to their target oligonucleotide probes on the microarray slide is performed by a microarray scanner device.

41. The method according to claim 36 wherein the step of measuring the intensity of fluorescence of cDNA or mRNA bound to their target oligonucleotide probes on the microarray slide is performed by a microarray scanner device.

42. The method according to claim 35 wherein the step of measuring the intensity of the hybridized labelled cDNA or mRNA bound to their target oligonucleotide probes on the dot blot is performed by a densitometric or fluorescent scanner device.

43. The method according to claim 36 wherein the step of measuring the intensity of the hybridized labelled cDNA or mRNA bound to their target oligonucleotide probes on the dot blot is performed by a densitometric or fluorescent scanner device.

44. The method according to claim 35 wherein the step of measuring the measuring the quantity of amplified cDNA is performed by a real-time quantitative PCR device.

45. The method according to claim 36 wherein the step of measuring the measuring the quantity of amplified cDNA is performed by a real-time quantitative PCR device.

46. The method according to claim 35 wherein the step of classifying the cancer subtype comprises the use of a computer.

47. The method according to claim 36 wherein the step of classifying the cancer subtype comprises the use of a computer.

48. The method according to claim 35 wherein the step of classifying the cancer subtype comprises a step of inputting computer readable instructions into a computer for any one or more of:
(a) performing unsupervised consensus clustering analysis of the glycosyltransferase gene expression profile from the subject test sample having one of the six cancer types compared with the glycosyltransferase gene expression profiles from a set of reference or training glycosyltransferase genes from subtypes of the corresponding cancer to that of the subject;
(b) calculating the distance of the gene expression profile of the subject test sample to each of the set of glycosyltransferase gene expression centroids from subtypes of the corresponding cancer to that of the subject; and
(c) classifying the subject test sample as a cancer subtype wherein the cancer subtype is classified as that corresponding to the nearest glycosyltransferase gene expression centroid compared with the glycosyltransferase gene expression profile from the subject test sample.

49. The method according to claim 36 wherein the step of classifying the cancer subtype comprises a step of inputting computer readable instructions into a computer for any one or more of:
(a) performing unsupervised consensus clustering analysis of the glycosyltransferase gene expression profile from the subject test sample having one of the six cancer types compared with the glycosyltransferase gene expression profiles from a set of reference or training glycosyltransferase genes from subtypes of the corresponding cancer to that of the subject;
(b) calculating the distance of the gene expression profile of the subject test sample to each of the set of glycosyltransferase gene expression centroids from subtypes of the corresponding cancer to that of the subject; and
(c) classifying the subject test sample as a cancer subtype wherein the cancer subtype is classified as that corresponding to the nearest glycosyltransferase gene expression centroid compared with the glycosyltransferase gene expression profile from the subject test sample.
